(19) United States
(12) Patent Application Publication
(10) Pub. No.: US 2014/0273222 A1
(43) Pub. Date: Sep. 18, 2014

(54) BIOREACTORS FOR TUBULAR ORGANS

(71) Applicants: Paul Weinberger, Augusta, GA (US); Hunter Faircloth, Savannah, GA (US); Donald Mettenburg, Augusta, GA (US); Frederick Rueggeberg, Augusta, GA (US)

(72) Inventors: Paul Weinberger, Augusta, GA (US); Hunter Faircloth, Savannah, GA (US); Donald Mettenburg, Augusta, GA (US); Frederick Rueggeberg, Augusta, GA (US)

(73) Assignee: GEORGIA REGENTS RESEARCH INSTITUTE, INC., Augusta, GA (US)

(21) Appl. No.: 14/216,697

(22) Filed: Mar. 17, 2014

Related U.S. Application Data
(60) Provisional application No. 61/794,938, filed on Mar. 15, 2013.

(51) Int. Cl. C12M 3/00; C12N 5/00

(52) U.S. Cl. CPC C12M 21/08; C12N 5/0062
USPC 435/395; 435/289.1; 435/293.1

(57) ABSTRACT

In one embodiment, a bioreactor includes a cartridge adapted to deliver growth media to inner and outer surfaces of a tubular organ scaffold, the cartridge having a container that includes a first passage adapted to deliver a first growth medium to the inner surfaces of the tubular organ scaffold and a second passage adapted to deliver a second growth medium to the outer surfaces of the scaffold, wherein the first and second growth media are different media.

### CROSS-REFERENCE TO RELATED APPLICATION(S)

This application claims priority to co-pending U.S. Provisional Application Ser. No. 61/794,938, filed Mar. 15, 2013, which is hereby incorporated by reference herein in its entirety.

### BACKGROUND

In 2008, a human trachea was grown from an adult patient's stem cells and the world's first tissue-engineered trachea was successfully transplanted. The trachea was grown by stripping an appropriately-sized donor trachea of its donor cells to form an extracellular scaffold. After the scaffold was formed, it was seeded with epithelial cells and chondrogenic mesenchymal stem cells from the patient. A basic bioreactor was used for the seeding process and an implantable trachea was formed in about 96 hours. The trachea was then successfully transplanted in the patient, and there were no signs of anti-donor antibodies in the four-month follow up. The patient appears to be living a normal life with the new trachea and has not seen any problems with rejection.

As is apparent from the above-described case, a trachea can be grown from a patient's own cells and successfully transplanted. There are problems, however, with the procedure that was used. Specifically, there are problems with the type of bioreactor that was used to grow tissue on the trachea scaffold. First, this type of bioreactor does not enable the growth media that are delivered to the inner and outer surfaces of the scaffold to be monitored or changed during incubation. This situation is disadvantageous because the cells in the media begin to die at a point at which the further tissue growth may be beneficial. Second, the bioreactor is not amenable to sterilization, which is required if it is to be reused. Third, the bioreactor design does not lend itself to mass production, which is required if the bioreactor and the procedure are to achieve widespread commercial use.

In view of the above discussion, it can be appreciated that it would be desirable to have an improved bioreactor for the growth of implantable tubular organs, such as the trachea.

### BRIEF DESCRIPTION OF THE DRAWINGS

The present disclosure may be better understood with reference to the following figures. Matching reference numerals designate corresponding parts throughout the figures, which are not necessarily drawn to scale.

FIG. 1A is a perspective view of an embodiment of a tubular organ bioreactor.

FIG. 1B is an exploded view of the tubular organ bioreactor of FIG. 1A.

FIG. 2 is a perspective view of a first end cap of a bioreactor cartridge of the bioreactor of FIG. 1.

FIG. 3 is a first perspective view of a medial separator of the bioreactor cartridge of the bioreactor of FIG. 1.

FIG. 4 is a second perspective view of the medial separator of the bioreactor cartridge of the bioreactor of FIG. 1.

FIG. 5 is an image of a prototype bioreactor cartridge that was fabricated for testing purposes.

### DETAILED DESCRIPTION

As described above, it would be desirable to have an improved bioreactor for the growth of implantable tubular organs that does not suffer from the drawbacks associated with current designs. Disclosed herein are tubular organ bioreactor designs that are adapted for growing user-definable tissue on both the inner and outer sides of a tubular organ scaffold. The organ can comprise any tubular organ, such as the trachea, the esophagus, or a blood vessel. In some embodiments, the bioreactor has a modular design in which an independent, disposable bioreactor cartridge is removably received by a drive station that is adapted to rotate the cartridge at a low speed. The bioreactor cartridge includes two ports that enable two different types of growth media to be simultaneously supplied to the scaffold and that further enable the media to be circulated and/or replaced during incubation. In some embodiments, the bioreactor cartridge facilitates fluid circulation of the growth media within the cartridge using passive circulation elements that naturally circulate the media when the cartridge is rotated.

In the following disclosure, various embodiments are described. It is to be understood that those embodiments are example implementations of the disclosed inventions and that alternative embodiments are possible. All such embodiments are intended to fall within the scope of this disclosure.

FIGS. 1A and 1B illustrate an embodiment of a tubular organ bioreactor 10. The bioreactor 10 has a modular design in which an independent, disposable bioreactor cartridge 12 is removably received by a drive station 14 that is adapted to rotate the cartridge at a low speed, such as approximately 1 rpm. The rotational speed of the cartridge 12 can be precisely controlled to provide the necessary shearing forces that aid in cell adhesion and differentiation. As shown in the figures, the bioreactor cartridge 12 generally comprises a container that includes an elongated cylindrical tube 16 having a first end 18 and a second end 20. In some embodiments, the tube 16 is transparent so that the interior of the cartridge 12 can be viewed by an operator and light used to perform measurements can enter and escape the cartridge to provide non-invasive analysis of tubular components in-situ. By way of example, the tube 16 is made of a transparent polymeric material. Provided at the second end 20 of the tube 16 is a gear 22 having a diameter that is larger than that of the tube. The gear 22 can be used to rotate the cartridge 12 during incubation.

As is shown most clearly in the exploded view of FIG. 1B, the drive station 14 includes a frame 26 that supports a drive motor 28 that drives a further gear 30, which is mounted to a shaft of the motor (not visible in the figure). In some embodiments, the motor 28 is an electric motor whose speed of rotation can be controlled with high accuracy. Also supported by the frame 26 are support shafts 32 that include idler wheels 34, which are adapted to contact and support the tube 16 of the bioreactor cartridge 12. During incubation, the drive motor 28 drives the gear 30, which in turn drives the gear 22 of the bioreactor cartridge 12 so as to rotate the cartridge.

As shown in FIG. 1A, a first end cap 36 can be inserted into the first end 18 of the bioreactor cartridge tube 16 to seal that end of the tube. FIGS. 1B and 2 show the first end cap 36 separate from the bioreactor cartridge 12. As illustrated in these figures, the first end cap 36 is a generally cylindrical member that includes an inside surface 38 that faces the center of the bioreactor cartridge 12 and an outside surface 40 that faces away from the center of the cartridge. By way of example, the first end cap 36 is made of a polymeric material. Provided on the inside surface 38 is a port 42 that is in fluid communication with an internal passage 44 that extends through the first end cap 36. With such a construction, fluid, such as a first growth medium that is used to seed the inner side of a tubular organ scaffold, can be delivered via the passage 44 into (and out of) the bioreactor cartridge 12. As is shown in FIGS. 1B and 2, the passage 44 can extend along a radial direction of the inside surface 38 of the first end cap 36.

With reference again to FIG. 1A, the bioreactor cartridge 12 further includes a second end cap 46 that can be inserted into the second end 20 of the bioreactor cartridge tube 16 to seal that end of the tube. As shown in FIG. 1B, the second end cap 46 can be connected to the gear 22. In some embodiments, the second end cap 46 is unitarily formed with the gear 22 from the same piece of material, such as a polymeric material. The second end cap 46 has a configuration similar to the first end cap 36. Accordingly, the second end cap 46 is a generally cylindrical member that includes an inside surface 48 that faces the center of the bioreactor cartridge 12.

Provided on the inside surface 48 of the second end cap 46 is a port 50 that is in fluid communication with an internal passage (not visible) that extends through the end cap and, if it is unitarily formed with the gear 22, through the gear. With such an arrangement, a fluid, such as a second growth medium that is used to seed the outer surface of a tubular organ scaffold, can be delivered through the passage into (and out of) the bioreactor cartridge 12. As shown most clearly in FIG. 1B, extending from the inside surface 48 toward the center of the bioreactor cartridge 12 is a mounting hub 52. During use, the mounting hub 52 supports an end of the tubular organ scaffold, which can be secured (e.g., with a suture or clamp) to the hub in a fluid-tight manner. In some embodiments, the mounting hub 52 includes a depression 53 at its tip that is adapted to receive the tip of a manifold described below.

As shown in FIG. 1A, a medial separator 54 can be positioned within the bioreactor cartridge tube 16 between the two end caps 36, 46. When so positioned, the medial separator 54 forms a seal with the tube 16 and divides it into two compartments, including a first compartment 56 and a second compartment 58, that are spaced from each other along the length of the tube 16. FIGS. 3 and 4 are detail views of the medial separator 54. As shown in these figures, the separator 54 also comprises a generally cylindrical member, which can be made of a polymeric material. With particular reference to FIG. 3, the separator 54 includes a first side 60 that faces the first end cap 36. Provided on the first side 60 are passive circulation elements 62 that are adapted to circulate the first growth medium when the elements are immersed in the medium and the bioreactor cartridge 12 is rotated in a direction identified by the dashed arrow. In the illustrated embodiment, the circulation elements 62 comprise planar impellers that extend out from first side 60 at an acute angle. In other embodiments, bucket-like elements that move the medium with the assistance of gravity can be used. Formed on high pressure sides of the impellers are inlets 64 (only one inlet visible in FIG. 3) through which the first growth medium can enter the separator 54. Formed on low-pressure sides of the impellers are outlets 66 (only one outlet visible in FIG. 3) through which the first growth medium can exit the separator 54. As described below, the inlets 64 are in fluid communication with one or more inlet passages (not visible) that extend through the separator 54, and the outlets 66 are in fluid communication with one or more outlet passages (not visible) that also extend through the separator.

Referring next to FIG. 4, the medial separator 54 further includes a second side 68 that faces the second end cap 46. Extending from the second side 68 is a central mounting hub 70 that, like the mounting hub 52, supports an end of the tubular organ scaffold, which can be secured to the hub in a fluid-tight manner. The aforementioned inlet passage(s) and outlet passage(s) extend through the mounting hub 70. In addition, the inlet passage(s) extend through a tubular manifold 74 that extends from the tip of the mounting hub 70. This manifold 74 can be used to deliver the second growth medium to the inner side of a tubular organ scaffold when it is mounted to the hubs 52, 70. FIG. 1A illustrates such mounting with a clear plastic tube 76 representing a scaffold that surrounds the manifold 74 and whose ends overlap the hubs 52, 70 of the second end cap 46 and the medial separator 54, respectively. As is apparent from FIG. 1A, when a scaffold (represented by tube 76) is supported by the hubs 52, 70, its inner surfaces define an inner chamber in which the first growth medium circulates and its outer surface defines an outer chamber in which the second growth medium can circulate. Returning to FIG. 4, the manifold 74 includes multiple openings 78 through which the first growth medium (supplied by the inlet passage(s) of the medial separator 54) can pass. The first growth medium can then travel between the manifold 74 and the inner surfaces of the scaffold, as indicated by the arrows, to return to the separator 54 and pass through its outlet passage(s). In this manner, the first growth medium can circulate between the first compartment 56 and the inner chamber defined by the scaffold.

To grow a tubular organ, such as a trachea, an appropriately-sized donor organ is obtained and is stripped of its donor cells to form an extracellular scaffold. Next, appropriate growth media can be prepared, which are to be separately applied to the inner and outer surfaces of the scaffold during the incubation process. In the case of the trachea, the first growth medium can be an epithelial cell growth medium suited for the inner surfaces of the scaffold, and the second growth medium can be a chondrogenic mesenchymal stem cell growth medium suited for the outer surfaces of the scaffold.

Once the growth media have been prepared, the prepared scaffold can be attached to the hubs 52, 70 of the medial separator 54 and the second end cap 46. This attachment can be achieved by separating the medial separator 54 from the second end cap 46 as shown in FIG. 1B, securing one end of the scaffold to one of the hubs 52, 70, connecting the medial separator and the second end cap together so that the tip of the manifold 74 is received by the depression 53 provided the tip of the hub 52, and securing the other end of the scaffold to the other hub. When this procedure is performed, the scaffold will surround the manifold 74 and its ends will be secured to the hubs 52, 70 in a fluid-tight manner.

Next the bioreactor cartridge 12 can be assembled by positioning the first end cap 36, medial separator 54, and second end cap 46 inside the cylindrical tube 16 in the configuration shown in FIG. 1A. Once assembled, the bioreactor cartridge 12 can be placed on the drive station 14 and the motor 28 can be used to rotate the cartridge about its longitudinal axis at a pre-defined, controllable speed.

The first compartment 56 of the bioreactor cartridge 12 can be filled to an appropriate degree with the first growth medium. In some embodiments, the first compartment 56 can be filled halfway with the first growth medium so that approximately half of its volume is occupied by the first growth medium. FIG. 5 is an image of a fabricated prototype bioreactor cartridge whose first compartment has been filled in this manner with a colored fluid that represents the first growth medium. If the volume of the first growth medium provided in the first compartment 56 is sufficient to supply the inner surfaces of the scaffold with all the cells it needs to complete the growth process, the first end cap 36 can be sealed so that no growth medium can enter or exit the first compartment. In embodiments in which it is desired to supplement or replace the first growth medium (e.g., to provide fresh cells to the scaffold), however, the first growth medium can be delivered to and from the first compartment 56 via the internal passage formed in the first end cap 36.

As the bioreactor cartridge 12 rotates, the passive circulation elements 62 drive the first growth medium contained in the first compartment 56 through the medial separator 54 and its manifold 74 so that the medium is delivered to the inner chamber defined by the scaffold. This is depicted in FIG. 5, which shows a clear plastic tube presenting the scaffold being halfway filled with the colored fluid. As the bioreactor cartridge 12 rotates, the inner surfaces of the scaffold are bathed in the first growth medium and the cells it contains. The first growth medium continuously circulates between the first compartment 56 and the inner chamber formed by the scaffold due to the presence of the passive circulation elements 62 and the passages formed through the medial separator 54. Because the volume of the first compartment 56 is much greater than the volume within the scaffold, the inner surfaces of the scaffold can be exposed to a greater number of cells than with previous bioreactors.

Simultaneous to bathing the inner surfaces of the scaffold with the first growth medium, the outer surfaces of the scaffold are bathed with the second growth medium. Specifically, the second growth medium can be delivered through the internal passage of the gear 22 and the second end cap 46 to the second compartment 58, which may also be referred to as the outer chamber defined by the scaffold. In some embodiments, the second compartment 58 can also be filled halfway so that approximately half of its volume is occupied by the second growth medium. In some embodiments, the second growth medium can be circulated into and out of the second chamber 56 via the internal passage of the gear 22 and cap 46 to ensure that fresh cells are provided to the scaffold as long as needed to complete the growth process.

After tissue has been grown on the inner and outer surfaces of the scaffold for an appropriate period of time (e.g., several days), the bioreactor cartridge 12 can be removed from the drive station 14 and disassembled to retrieve the implantable organ. At this point, the cartridge 12 can be discarded and the organ can be implanted in the patient.

Claimed are:

1. A bioreactor cartridge adapted to deliver growth media to inner and outer surfaces of a tubular organ scaffold, the cartridge comprising:
a container including a first passage adapted to deliver a first growth medium to the inner surfaces of the tubular organ scaffold and a second passage adapted to deliver a second growth medium to the outer surfaces of the scaffold, wherein the first and second growth media are different media.

2. The bioreactor cartridge of claim 1, wherein the container contains a first element having a first hub and a second element having a second hub, wherein the hubs face each other such that an end of the tubular organ scaffold can be secured to each of the hubs to define inner and outer chambers, the inner chamber being adapted to receive the first growth medium and the outer chamber being adapted to receive the second growth medium.

3. The bioreactor cartridge of claim 2, further comprising a manifold that extends from the first hub toward the second hub, the manifold being configured to deliver the first growth medium to the inner chamber.

4. The bioreactor cartridge of claim 3, wherein the first element comprises a separator that separates the container into first and second compartments, wherein the first compartment is adapted to contain a volume of the first growth medium and the second compartment contains the first and second hubs.

5. The bioreactor cartridge of claim 4, wherein the separator includes an inlet passage that connects the first compartment to the manifold and an outlet passage that connects the inner chamber to the first compartment, wherein the first growth medium can flow from the first compartment, through the inlet passage, out from the manifold and into the inner chamber, through the outlet passage, and back to the first compartment to enable circulation of the first growth medium between the first compartment and the inner chamber.

6. The bioreactor cartridge of claim 5, wherein the separator further includes passive circulation elements that drive the first growth medium between the first compartment and the inner chamber.

7. The bioreactor cartridge of claim 6, wherein the passive circulation elements comprise impellers that drive the first growth medium when the container is rotated.

8. The bioreactor cartridge of claim 2, wherein the second element is an end cap that seals an end of the container and the second passage extends through the end cap.

9. The bioreactor cartridge of claim 8, further comprising a further end cap that seals an opposite end of the container, wherein the first passage extends through the further end cap.

10. A bioreactor cartridge comprising:
an elongated tube;
a first end cap that seals a first end of the tube;
a second end cap that seals a second end of the tube, the second end cap comprising a first hub that faces the first end cap; and
a medial separator provided in the tube between the first and second end caps having a second hub that faces the first hub of the second end cap, the separator dividing the tube into a first compartment and a second compartment, the first and second hubs being contained in the second compartment;
wherein the first and second hubs are adapted to receive opposite ends of a tubular organ scaffold to which growth media is to be applied, inner surfaces of the scaffold defining an inner chamber in which a first growth medium can circulate and outer surfaces of the scaffold defining an outer chamber in which a second growth medium can circulate.

11. The bioreactor cartridge of claim 10, wherein the elongated tube is transparent.

12. The bioreactor cartridge of claim 10, wherein the first end cap comprises a passage with which the first growth medium can be provided to the first compartment and the second end cap comprises a passage with which the second growth medium can be provided to the second compartment

13. The bioreactor cartridge of claim 10, wherein the medial separator comprises an inlet passage through which the first growth medium can travel to pass from the first compartment to the inner chamber and an outlet passage through which the first growth medium can travel to pass from the inner chamber to the first compartment.

14. The bioreactor cartridge of claim 13, wherein the medial separator further comprises a manifold that extends from the second hub, the manifold being adapted to deliver the first growth medium from the inlet passage to the inner chamber.

15. The bioreactor cartridge of claim 13, wherein the medial separator further includes passive circulation elements that drive the first growth medium through the inlet passage.

16. The bioreactor cartridge of claim 15, wherein the passive circulation elements comprise impellers that drive the first growth medium through the inlet passage when the container is rotated.

17. A bioreactor comprising:
a bioreactor cartridge including a first passage adapted to deliver a first growth medium to the inner surfaces of the tubular organ scaffold and a second passage adapted to deliver a second growth medium to the outer surfaces of the scaffold, wherein the first and second growth media are different media; and
a drive station that can removably receive the bioreactor cartridge, the drive station being adapted to rotate the bioreactor cartridge to circulate the growth media within the cartridge.

18. The bioreactor of claim 17, wherein the bioreactor cartridge comprises a first element having a first hub and a second element having a second hub, wherein the hubs face each other such that an end of the tubular organ scaffold can be secured to each of the hubs to define inner and outer chambers, the inner chamber being adapted to receive the first growth medium and the outer chamber being adapted to receive the second growth medium.

19. The bioreactor of claim 17, wherein the drive station includes an electric motor.

20. The bioreactor of claim 17, wherein the bioreactor cartridge and the drive station comprise meshing gears that enable rotation of the bioreactor cartridge.

21. A method for growing a tubular organ for implantation within a patient, the method comprising:
mounting ends of a tubular organ scaffold to opposed hubs within a bioreactor cartridge, inner surfaces of the scaffold defining an inner chamber and outer surfaces of the scaffold defining an outer chamber;
providing a first growth medium in a first compartment of the bioreactor cartridge;
connecting the first compartment with the inner chamber so that the first growth medium can travel between the first compartment and the outer chamber;
providing a second growth medium in the inner chamber; and
rotating the bioreactor cartridge so as to circulate the first growth medium between the first compartment and the inner chamber and to circulate the second growth medium in the outer chamber.

22. The method of claim 21, wherein the bioreactor cartridge comprises passive circulation elements that drive the first growth medium between the first compartment and the inner chamber when the bioreactor cartridge is rotated.
